(19) United States
(12) Patent Application Publication
(10) Pub. No.: US 2003/0176857 A1
(43) Pub. Date: Sep. 18, 2003

(54) Assembly for embolic treatments

(76) Inventor: Kyu Ho Lee, Seoul (KR)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/386,824

(22) Filed: Mar. 12, 2003

Related U.S. Application Data
(63) Continuation-in-part of application No. 09/757,408, filed on Jan. 9, 2001, now Pat. No. 6,558,377.

(30) Foreign Application Priority Data
Jul. 26, 2000 (KR) 2000-42929

(51) Int. Cl. A61B 18/18

(52) U.S. Cl. 606/32; 606/41; 606/108

(57) ABSTRACT

An assembly for embolization is provided, which includes a simplified member and an embolic material inducing an improved thrombus in vascular malformations. The assembly comprises an implant to be inserted within the target site; and a guiding member with its distal end coupled with the implant, for guiding the implant to the target site, wherein the guiding member is made of an electrically conducting material and includes a multiplicity of tapered configurations.

This application is a Continuation-In-Part of application Ser. No. 09/757,408, filed on Jan. 9, 2001.

### FIELD OF THE INVENTION

The present invention relates to the embolic treatment field, and more particularly to an assembly for embolization, which includes a simplified guiding member and an embolic material inducing an improved thrombus in vascular malformations.

### BACKGROUND OF THE INVENTION

Vascular malformations, such as a cerebral aneurysm, may be treated by an operative procedure, which includes putting a patient under general anesthesia to craniotomy, exposing the cerebral aneurysm in the patient using an operating microscope and a microsurgical unit, and clipping a cervical portion of the cerebral aneurysm with a particular metallic clip. However, such treatments suffer from drawbacks. They still involve considerable hazard and prolonged operating time, which in turn, may cause serious sequelae.

An alternative treatment that may be utilized is Minimal Invasive Treatment (MIT), which employs a technique disclosed in U.S. Pat. No. 5,122,136 issued to Guglielmi et al. and U.S. Pat. Nos. 4,884,579 and 4,739,768 issued to Engelson. MIT inserts an embolic material within vascular malformations, such as a cerebral aneurysm, through the use of a micro catheter and a guiding wire under fluoroscopy to occlude the vascular malformations. In contrast with the craniotomy treatment, MIT has the merits of allowing operation under a slight anesthesia within a short operation time, to thereby minimize serious sequelae and also lower operation cost.

An embolic material mainly utilized in MIT includes a metallic coil. The metallic coil is disclosed in, for example, U.S. Pat. Nos. 5,354,295, 5,669,905, and 6,066,133 and Japanese Patent Nos. 10-057385, 11-047138, and 11-076249. The metallic coil cited in U.S. Pat. No. 5,669,905 will be described below.

FIG. 1 is a pictorial view of a metallic embolic coil used in conventional MIT. As shown in FIG. 1, a guiding wire assembly 100 typically includes a stainless steel-based guiding wire 1 and a coil-shaped embolic material 8, wherein guiding wire 1 is tapered at its distal end and embolic material 8 is connected with the distal end of guiding wire 1 by micro welding. Embolic material 8 is made of a radiopaque material including Platinum, Tungsten, Iridium or these alloys, and has welded portions 6 and 7 at both its ends. Welded portions 6 and 7 are made of Platinum that act as markers under fluoroscopy.

A surface of guiding wire 1 is coated with an insulating material such as Teflon, with the exception of a proximal end 5 acting as a sacrificial link to be connected with welded portion 6 of embolic material 8. Sacrificial link 5 is made of an electrically conducting material such as stainless steel, which is a portion to be detached from guiding wire 1 by electrolytic disintegration. Guiding wire 1 is coupled with welded portion 6 of embolic material 8 via sacrificial link 5, which is interposed in a sleeve 2 and a plug 3 inserted within an internal coil 4. Internal coil 4 is designed to provide column strength to guiding wire 1, without negatively influencing the flexibility of a tapered portion in guiding wire 1. As shown in FIG. 1, embolic material 8 has been designed to change shape into a coil form when it is gradually withdrawn from a micro catheter (not shown), to thereby allow the embolic material to adapt to the shape of the vascular malformation.

FIGS. 2A and 2B are pictorial views illustrating insertion and detachment processes of embolic material 8 in the prior art. Typically, the insertion of embolic material 8 in a vascular malformation 11 is performed using fluoroscopy under local anesthesia. Specifically, as shown in FIG. 2A, an operator guides a micro catheter 10 near a neck 12 of vascular malformation 11 in a living being or a patient. After that, the operator inserts guiding wire 1 attached embolic material 8 on its distal end into micro catheter 10, and gently pushes guiding wire 1 using fluoroscopy at least until sacrificial link 5 is exposed beyond the distal end of micro catheter 10.

In an ensuing step, an electrical loop is formed wherein a positive electrode of a power supply 13 is attached to sacrificial link 5 of guiding wire 1 and a negative electrode is placed in electrical contact with the skin of the patient. Thereafter, power supply 13 is turned on to allow a direct current (DC) power with alternating current (AC) superposition to be applied to embolic material 8 through sacrificial link 5 of guiding wire 1. As a result of the above process, embolic material 8 is detached from guiding wire 1 by electrolysis as shown in FIG. 2B. Next, guiding wire 1 and micro catheter 10 are withdrawn from vascular malformation 11.

FIG. 3 shows a schematic block diagram of the prior art apparatus for detecting the detachment of embolic material 8 from guiding wire 1. An apparatus 200 according to the prior art includes a constant current source 16, a circuit 18 for detecting the detachment of embolic material 8, and a microprocessor 19. Constant current source 16, which includes an operational amplifier (OP Amp) 16a and a DC feedback loop 16b, provides a constant current to a patient 17. OP Amp 16a will oscillate at approximately 30 kHz at an amplitude of several hundred milli-volts due to a lagging error correction signal (out-of-phase feedback). That is, OP Amp 16a provides a DC current with AC superposition. The amplitude of such AC signal is dependent on bandwidth characteristics of OP Amp 16a, the AC impedance of the stainless steel and embolic material 8, and the patient's body. The DC constant current flowing out of OP Amp 16a flows through sacrificial link 5 of guiding wire 1 to embolic material 8.

Although sacrificial link 5 and embolic material 8 are physically connected in series, immersion of them in an electrolytic solution forms two parallel DC current paths, each of which is grounded through the body of patient 17. Specifically, by ion flow away from sacrificial link 5 during electrolysis, the DC current with AC superposition flowing between sacrificial link 5 and embolic material 8 in vascular malformation 11 is branched as follows. The majority of the DC current (above 99%) flows through sacrificial link 5 with the remaining (less 1%) flowing through embolic material 8. Thus, if embolic material 8 is separated from sacrificial link 5 and a portion of sacrificial link 5 remains attached to guiding wire 1, the main DC current is fed back to DC feedback loop 16b of constant current source 16. The AC current is grounded through embolic material 8.

As shown in FIG. 3, the DC current with AC superposition is blocked out by a pick-off capacitor (not shown). Only the AC signal is fed to detection circuit 18 for the measurement of AC impedance. Detection circuit 18 receives the AC current from embolic material 8 in patient 17 to detect whether or not embolic material 8 is detached. Specifically, the AC current fed to detection circuit 18 is amplified in an AC signal amplifier 18a and rectified in an AC-DC rectifier 18b. Then, the rectified DC signal is amplified in a DC level amplifier 18c and sent to microprocessor 19, wherein the amplified DC level is representative of the amplitude of the AC voltage outputted from OP Amp 16a.

Microprocessor 19 monitors the level of the amplified DC signal every 50 to 200 milliseconds and constantly averages the signal of every specific sample. In this manner, if a sudden DC voltage drop is incurred, microprocessor 19 determines that embolic material 8 has been detached from guiding wire 1.

In the prior art, OP Amp 16a oscillates on its own, which allowed the monitoring of the AC impedance fluctuation by detection circuit 18. However, since there are fluctuations in the self-oscillation signal flowing from unit to unit, it fails to exactly determine the instant the embolic material is detached. That is to say, a fluctuation in the AC impedance depends on a length of embolic material 8 and other physical factors, thereby resulting in poor detachment detection.

To support this, as shown in FIG. 4, an external AC source 20b is utilized to ensure all units will show the identical response to the fluctuation in the AC impedance. In FIG. 4, an AC source 20b is coupled with a reference input V_{ref} of an OP Amp 20a so as to modulate the output current of OP Amp 20a (i.e., provide AC superposition on the DC current). A DC current with AC superposition is outputted from OP Amp 20a and sent to embolic material 8 through sacrificial link 5 of guiding wire 1. As a result, two AC and DC current paths branch as described above with reference to FIG. 3. The DC current with AC superposition from patient 17 is fed back to an AC & DC feedback loop 20c of a constant current source 20 and fed to OP Amp 20a.

As stated above, the DC current with AC superposition is blocked out by a pick-off capacitor (not shown). Only the AC signal is fed to a detection circuit 21 for the measurement of AC impedance fluctuation. In detection circuit 21, since the amplitude of the AC signal is substantially greater than that of FIG. 3, DC level amplifier 18c in FIG. 3 is not necessary. As noted, the AC signal is amplified in an AC signal amplifier 21a in detection circuit 21 and rectified in an AC-DC rectifier 21b. Then, the rectified DC signal is sent to microprocessor 19.

In short, the prior art apparatuses previously disclosed detect the detachment of the embolic material using the AC signal. Accordingly, the prior art apparatuses suffer from a drawback that if a fluctuation in the AC impedance depends on the length of the embolic material and other physical factors, exactly detecting the detachment instant is difficult. In addition, the prior art guiding wire assembly for embolization demands an additional coil for maintaining the shape of the guiding wire and an additional signal source, thereby rendering the apparatus rather complex and costly. Likewise, although the embolic material in the prior art has been fabricated with Platinum, Tungsten, Gold, Iridium or these alloys for thrombus in vascular malformations, effectively enhancing the rate of thrombus without any application of high power to the material would be desirable.

### SUMMARY OF THE INVENTION

It is, therefore, an objective of the present invention to provide an assembly for embolization, which includes a simplified guiding member and an embolic material inducing an improved thrombus in vascular malformations.

In accordance with a preferred embodiment of the present invention, there is provided an assembly for use in occluding a target site in a living being, which comprises: an implant to be inserted within the target site; and a guiding member with its distal end coupled with the implant, for guiding the implant to the target site, wherein the guiding member is made of an electrically conducting material and includes a multiplicity of tapered configurations.

### BRIEF DESCRIPTION OF DRAWINGS

The above and other objects and features of the present invention will become apparent from the following description of preferred embodiments given in conjunction with the accompanying drawings.

FIG. 1 is a pictorial view of a metallic embolic coil used in a conventional Minimal Invasive Treatment (MIT).

FIGS. 2A and 2B show pictorial views illustrating insertion and detachment processes of an embolic material in the prior art, respectively.

FIG. 3 offers a schematic block diagram of the prior art apparatus for detecting the detachment of the embolic material from a guiding wire.

FIG. 4 depicts a schematic block diagram of the prior art apparatus using an external alternating current (AC) source.

FIGS. 5A to 5C are pictorial views of an embolic material in accordance with the present invention, respectively.

FIGS. 6A to 6C represent pictorial views of an assembly for embolization in which the embolic material having a platinum-based wire therein is coupled with a guiding wire in accordance with the present invention, respectively.

FIGS. 7A to 7E exhibit pictorial views of various guiding wires in accordance with the present invention, respectively.

FIG. 8 is a micro-envelope surrounding a portion at which a micro-welded portion and the embolic material are coupled.

FIG. 9 provides a schematic block diagram of an embolic material detachment detecting system in accordance with the present invention.

FIG. 10 denotes a detailed block diagram of a constant current source and a current-sensing block shown in FIG. 9.

FIG. 11 designates a change in current in a sacrificial link during the electrolysis.

FIG. 12 illustrates current paths flowing through the sacrificial link and the embolic material, during the application of a current to the embolic material.

FIG. 13 is a flow chart that will be used to describe a method for detecting the detachment of the embolic material in accordance with the present invention.

FIG. 14 presents a graphical representation illustrating a fluctuation in voltage in the sacrificial link during the electrolysis.

FIGS. 15A to 15C are various types of tubes used in keeping therein the assembly for embolization with the embolic material and the guiding wire in accordance with the present invention.

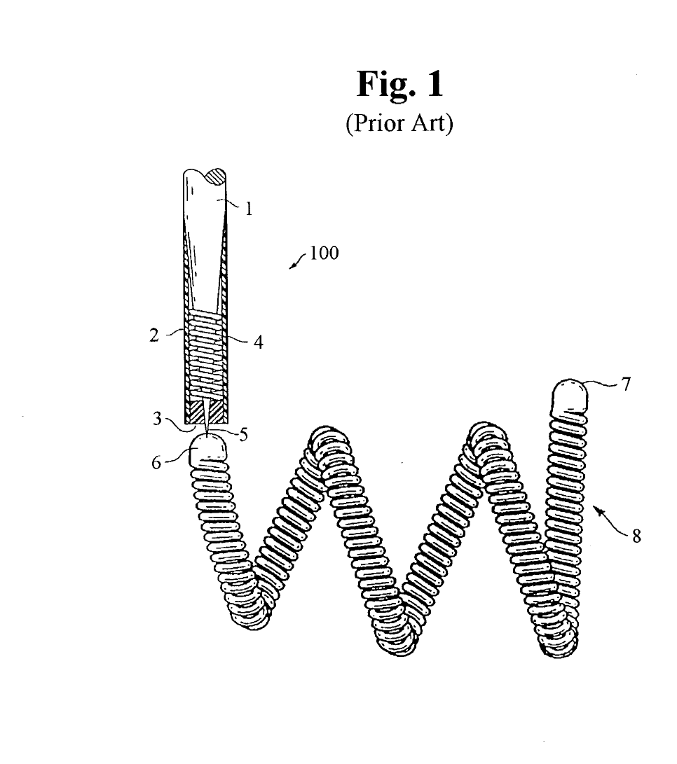

### DETAILED DESCRIPTION OF THE PRESENT INVENTION

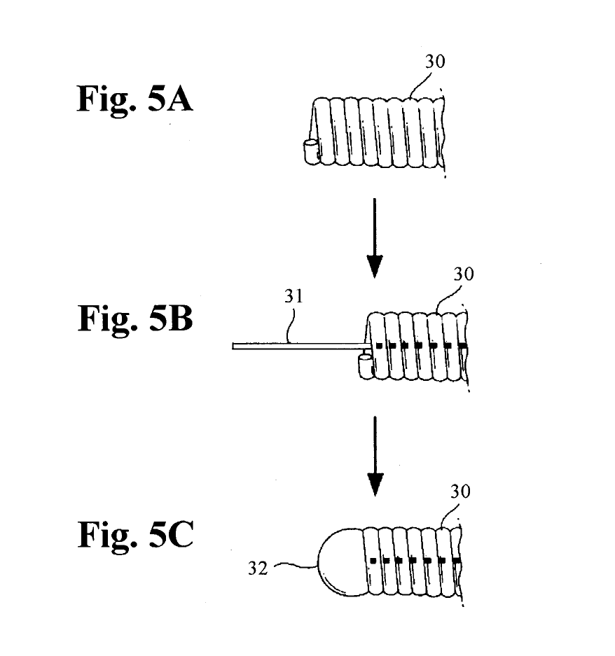

Referring to FIGS. 5A to 5C, pictorial views of an embolic material in accordance with a preferred embodiment of the present invention are shown. As shown in FIG. 5A, a primary embolic material 30 is fabricated by forming Tungsten or Iridium alloy wires with the main part of Platinum, in a coil fashion, for example. In this case, the diameter of the wire is 25˜75 μm and the inner diameter of primary embolic material 30 (hereinafter, called embolic coil 30) is, e.g., 100˜150 μm. In this case, the mixture ratio of Platinum and Iridium may be preferably 95 to 5 or 85 to 15 wt. % and Platinum and Tungsten may be preferably 92 to 8 wt. %.

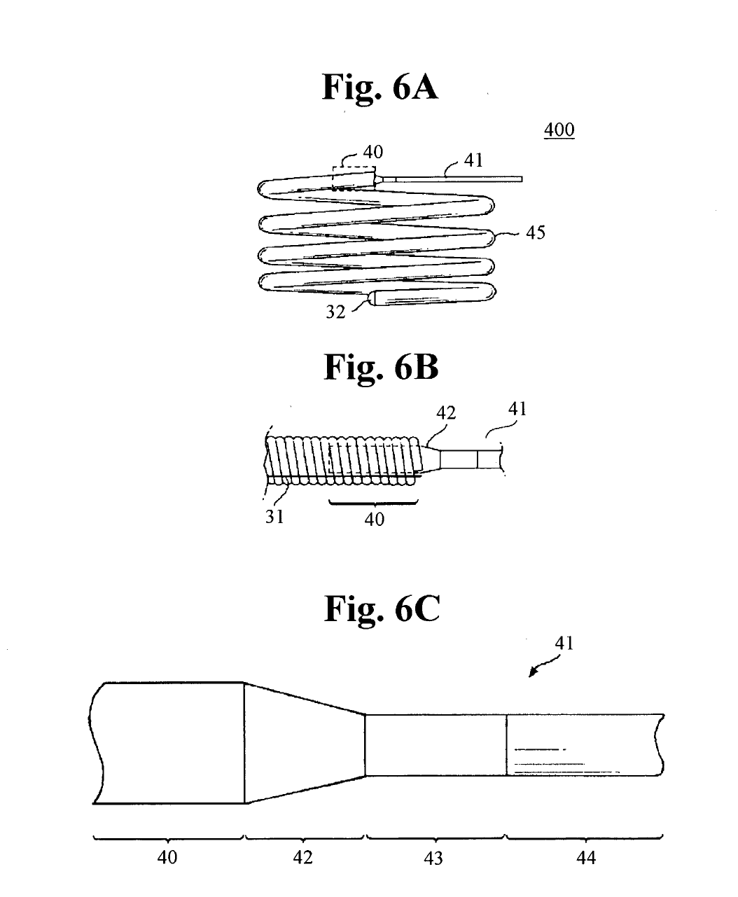

As shown in FIG. 5B, a pure Platinum-based wire 31 having 15 μm diameter is inserted into embolic coil 30. After that, by melting Platinum-based wire 31 at one end of embolic coil 30, a semicircle-like welded portion 32 is formed as presented in FIG. 5C. Welded portion 32 functions as a marker during the insertion of embolic coil 30 into a target site in a living being or organism under fluoroscopy. Embolic coil 30 formed thus is modified in a further coil fashion to allow it to be adaptively transformed to a shape of the vascular malformation, as shown in FIG. 6A. In FIG. 6A, a secondary embolic coil 45 has a diameter of 2˜8 mm and a length of 4˜20 cm, for example. Although the shape of secondary embolic coil 45 is cylindrical, it may be conic or waveform, which can be adaptively transformed in response to the shape of the vascular malformation. For the purpose of this specification, secondary embolic coil 45 is hereinafter referred to as embolic material 45. Heating it in a temperature of approximately 600˜800° C., preferably 640˜750° C., for about 30 minutes, under the vacuum condition of 1 atmospheric pressure, and then performing a rapid air-cooling may form embolic material 45.

As is well known, Platinum, Iridium, and Tungsten have excellent conductivity and radiopaque characteristics. In the above, although a Tungsten or Iridium alloy with the main part of Platinum has been used as a source of embolic material 45, any material with excellent conductivity, radiopaque characteristic, and biocompatibility may be used. Major reason why Platinum-based wire 31 is inserted within embolic material 45 is to: function as a thermoseed during a radio-frequency heating to accelerate the thrombus; increase the effect of heat generation and the column strength of embolic material 45; improve the plasticity of embolic coil 30; and prevent its decomposition during electrolysis. In addition, Nickel-Titanium alloys with superior plasticity and flexibility may be used, especially in the case of radiofrequency inductive heating for enhancing thrombosis within the aneurysm.

Referring to FIGS. 6A to 6C, pictorial views of an assembly for embolization are shown in which embolic material 45 having Platinum-based wire 31 therein is coupled with a guiding wire in accordance with the present invention. As shown in FIG. 6A, the proximal end of embolic material 45 and the distal end of a guiding wire 41 are connected together by inserting guiding wire 41 approximately 0.2˜0.3 mm into embolic coil 30 (shown in FIGS. 5A to 5C).

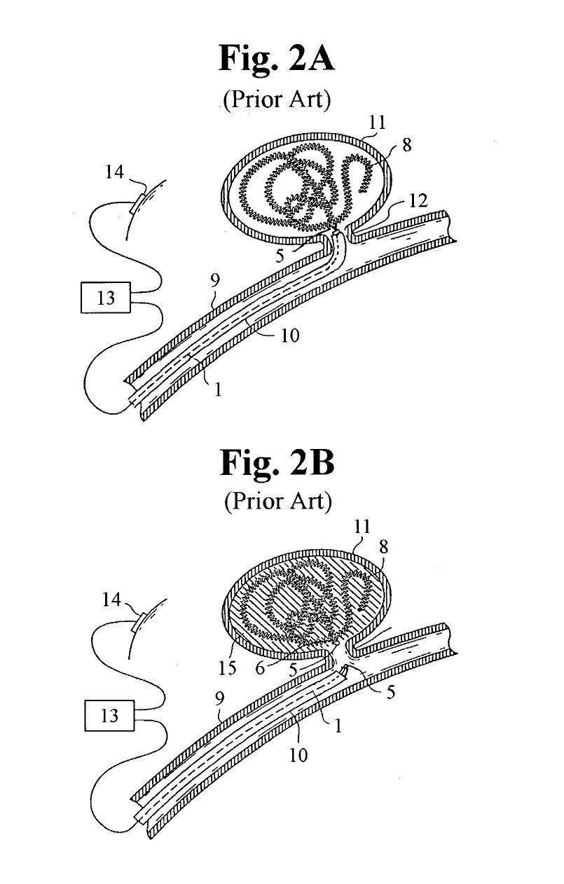

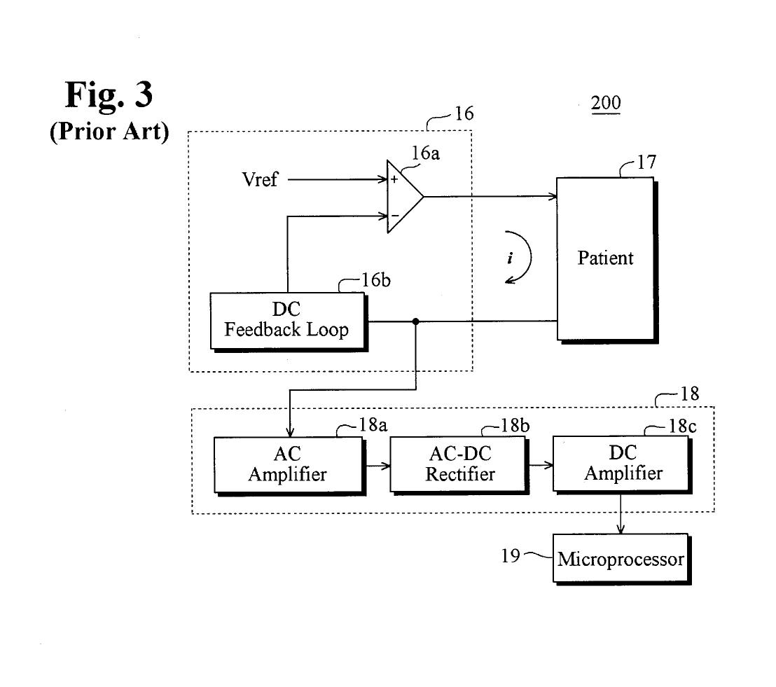

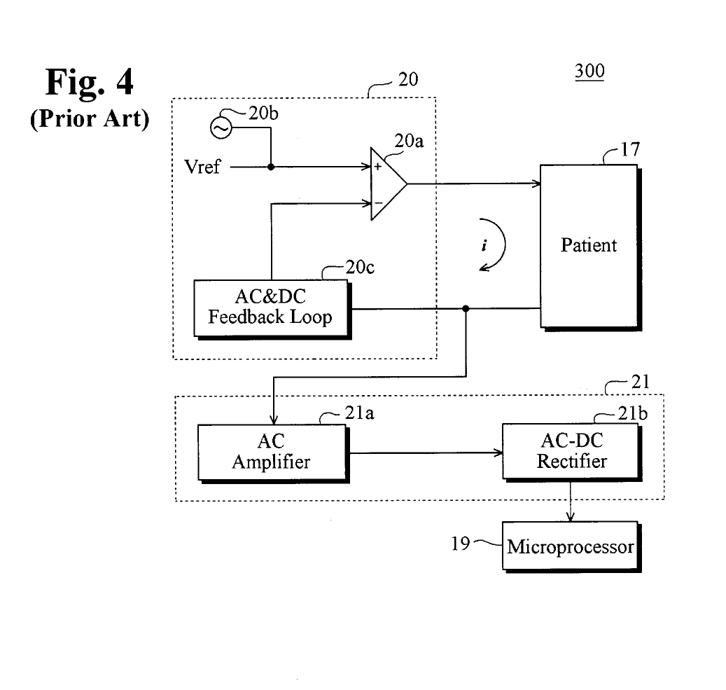

As shown in FIG. 6B, the diameter of a distal end 40 of guiding wire 41 is slightly less than the internal diameter of embolic coil 30. The connection is made by resistive micro-welding. In this case, the condition of the micro-welding is set such that a change in resistance after the micro-welding should be in the range of 0.02˜0.03 ohm. The micro-welding is performed at a contact surface between embolic material 45 and guiding wire 41 without using a welding flux. Guiding wire 41, made of an electrically conducting material such as stainless steel, is used to guide embolic material 45 to vascular malformation 11 shown in FIGS. 2A and 2B.

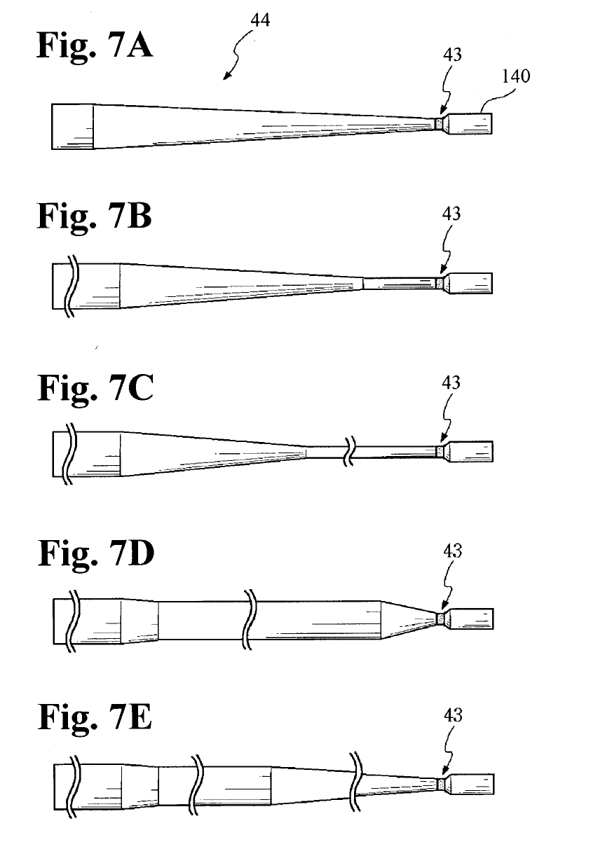

As shown in FIG. 6C, it consists of distal end 40 (hereinafter, referred to connection portion 40), a tapered portion 42, a sacrificial link 43, and a body 44. Body 44 is coated with a PTFE (Poly Teflon Fluorine Ethylene) material with good hydrophilicity and high insulating properties, and low frictional force at a thickness of approximately 10 μm. About a 0.01 inch length of tapered portion 42 and about 0.008 or 0.01 inch length of sacrificial link 43 are exposed without an insulating coating so that these can be dissolved during electrolysis. Disconnection within guiding wire 41 mainly occurs at sacrificial link 43 during electrolysis. Referring to FIGS. 7A to 7E, pictorial views of various guiding wires are shown in accordance with a preferred embodiment of the present invention. As shown in FIGS. 7A to 7E, body 44 is tapered in different fashions toward sacrificial link 43 to allow it to be easily inserted within micro catheter 10 and to be adaptively transformed according to a shape of blood vessel.

As mentioned above, in accordance with the assembly for embolization of the present invention, guiding wire 41 does not require a support coil as was provided in the prior art for providing the column strength to guiding wire 41, thereby making it possible to simplify the structure of the assembly for embolization.

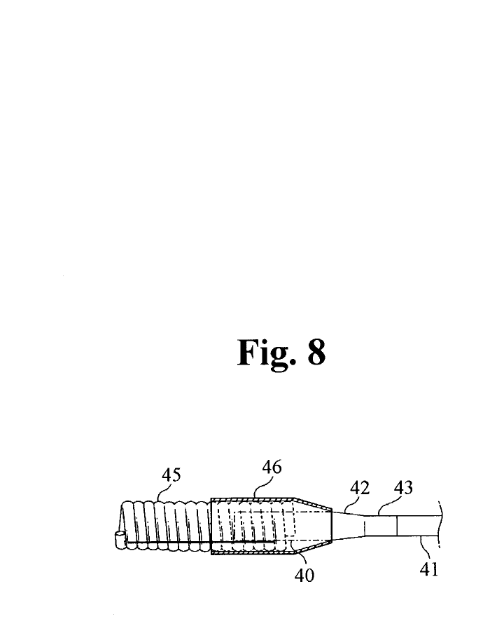

In accordance with the present invention, as shown in FIG. 8, a micro-tube 46 having a cap-like or envelope shape may be provided over connection portion 40. Specifically, micro-tube 46 is made of a pure radiopaque Gold or a Platinum alloy less than 10 μm in thickness and about 0.3˜0.5 mm in length, and may be narrowed in the direction of guiding wire 41. Micro-tube 46 helps embolic material 45 to smoothly exit from the distal end of micro catheter 10 (shown in FIGS. 2A and 2B). Micro-tube 46 made of the pure radiopaque gold or platinum alloy functions as a marker for the position of embolic material 45 in fluoroscopy using, e.g., X-ray. In addition, micro-tube 46 improves the positioning of embolic material 50 when embolic material 50 is inserted into the aneurysm.

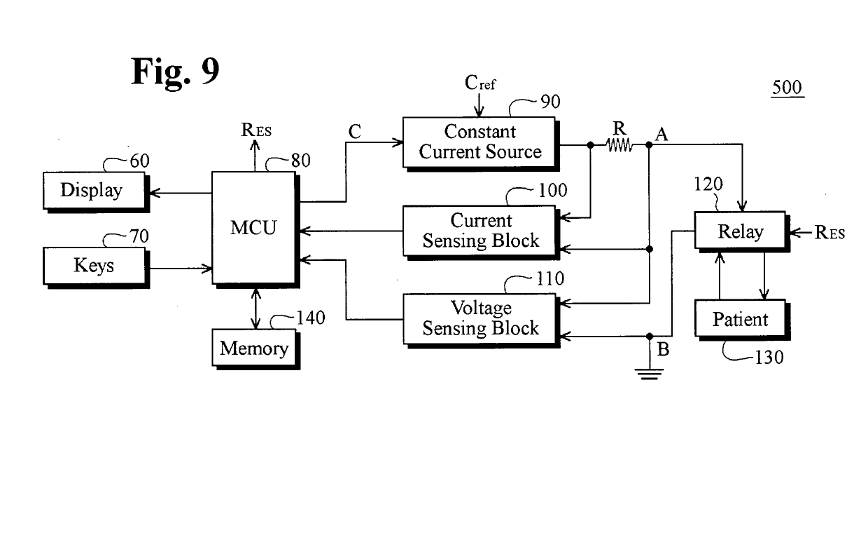

FIG. 9 is a block diagram of an embolic material detachment detecting system in accordance with the present invention. As shown in FIG. 9, an embolic material detachment detecting system 500 of the present invention comprises a display 60, function keys 70, a micro-controller unit (MCU) 80, a constant current source 90, a current-sensing block 100, and a voltage-sensing block 110. System 500 incorporates a power source such as a battery (not shown), e.g., a DC 9V power supply. Function keys 70 and display 60 are respectively used to set and display currents, voltages, and electrolysis time. Constant current source 90 is a typical circuit that generates and provides a 2 mA constant current to a patient 130 through a relay 120 under the control of a reference current C_{ref} and MCU 80.

An insertion process of the inventive assembly for embolization into patient 130 is similar to that of the prior art described with reference to FIGS. 2A and 2B, and therefore further description thereof is omitted.

First, in order to electrically detach embolic material 45 positioned within a vascular malformation such as the aneurysm from guiding wire 41, a positive electrode of the power supply (not shown) is attached to the proximal end of guiding wire 41 and a negative electrode thereof is placed in electrical contact with the skin of patient 130. In this situation, a constant current of 1˜2 mA is applied to patient 130. Specifically, when the power supply is turned on, relay 120 is rendered conductive in response to an enable signal R_{ES} provided thereto from MCU 80, resulting in a closed loop including system 500 and patient 130. That is, the positive current, which is applied to guiding wire 41 coupled with relay 120, flows to embolic material 45 and Platinum-based wire 31 inserted therein via sacrificial link 43.

Current-sensing block 100 continuously senses a current across a resistor R and provides a sensed current to MCU 80. In response to the sensed current from current-sensing block 100, MCU 80 provides a current-level control signal C to constant current source 90 so as to allow it to continuously generate the constant current of 1˜2 mA.

When relay 120 is in a conductive state, voltage-sensing block 110 continuously senses the fluctuation in impedance of sacrificial link 43 by detecting a difference in voltage between nodes A and B in FIG. 9. The sensed voltage by voltage-sensing block 110, i.e., an impedance value, is forwarded to MCU 80. Specifically, if the constant current of 1˜2 mA is provided to sacrificial link 43 inserted into patient 130 for a predefined time period, a minute electrolysis occurs at embolic material 45 made of an Iridium (or Tungsten) alloy with the main part of Platinum, but the majority occurs at sacrificial link 43 on which no insulating material is coated. In such event, voltage-sensing block 110 senses minute changes in the DC impedance of the distal end of guiding wire 41 and provides it to MCU 80.

MCU 80 determines whether or not sacrificial link 43 has been disconnected from guiding wire 41, based on the sensed current value received from current-sensing block 100 and the impedance change received from voltage-sensing block 110. After sacrificial link 43 is disconnected, MCU 80 activates an alerting device such as a beeper (not shown) to inform the operator of the detachment of sacrificial link 43. Simultaneously, MCU 80 renders relay 120 non-conductive to prevent an undesirable current from being applied to patient 130, and renders constant current source 90 non-operative. MCU 80 also displays on display 60 currents and voltages detected at the moment of detachment.

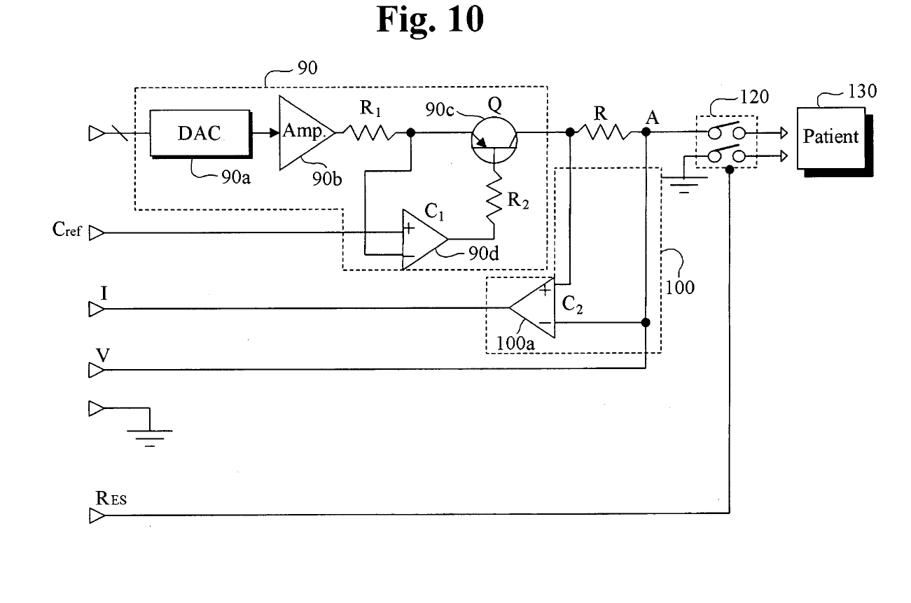

FIG. 10 is a detailed block diagram of constant current source 90 and current-sensing block 100 shown in FIG. 9. As stated above, in response to the enable signal R_{ES} from MCU 80, relay 120 is rendered conductive to form an electrical connection between patient 130 and system 500 of the present invention. As shown in FIG. 10, constant current source 90 includes a digital-to-analog converter (DAC) 90a, an OP Amp 90b, registers R1 and R2, a transistor Q 90c, and a comparator C1 90d. Constant current source 90 provides the constant current of 1˜2 mA during the operation, based on the reference current C_{ref} and a current signal from a power supply (not shown). Constant current source 90 allows the current applied to patient 130 to be increased after a delay, e.g., t₁ to t₂, as shown in FIG. 11, thereby making it possible to protect patient 130 from a sudden current application, which could cause an electrical shock. Current-sensing block 100 senses the current across the resistor R by using a comparator C2 100a and generates the sensed current I.

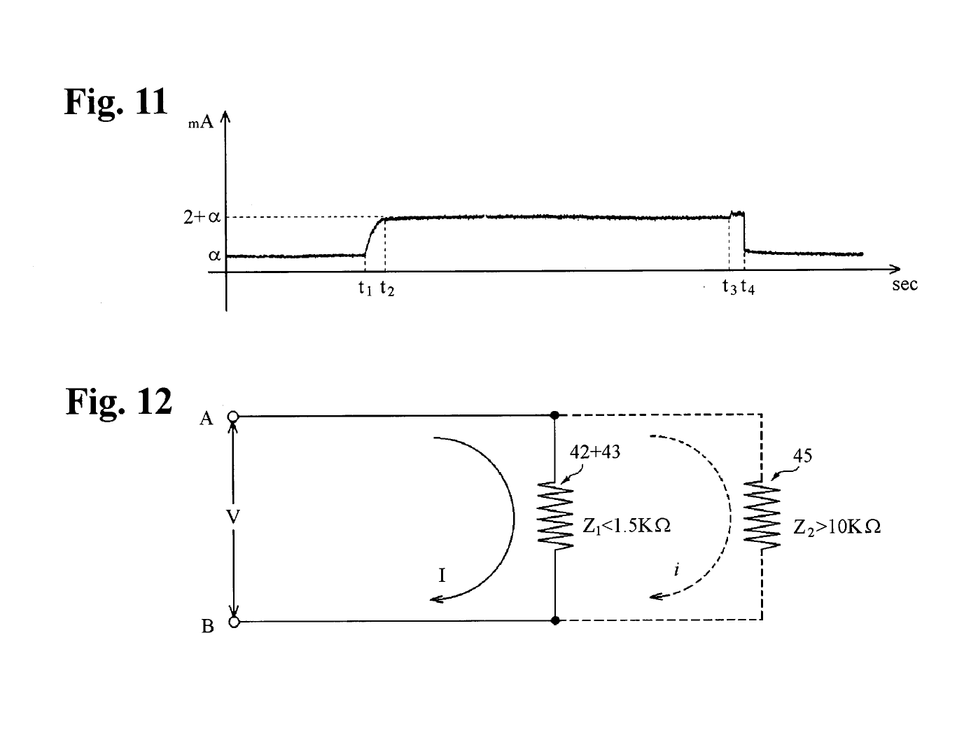

Applying the current, an electrolytic action occurs at embolic material 45 and sacrificial link 43 of guiding wire 41 inserted in vascular malformation 11. As is well known, a minute electrolysis occurs with Platinum with no chemical reaction, but the stainless steel is subject to the electrolysis. Specifically, since impedance Z1 of stainless steel-based sacrificial link 43 (including tapered portion 42) is less than about 1 kω, and impedance Z2 of Platinum-based embolic material 45 is larger than about 2 kω, the majority of current flows across sacrificial link 43, as indicated by a solid line in FIG. 12. Accordingly, a difference in voltage between nodes A and B nearly corresponds to the impedance change of sacrificial link 43 so that exactly determining the instant that sacrificial link 43 is detached is possible.

When relay 120 is in an conductive state, i.e., an electrical connection is formed between patient 130 and system 500, the voltage in node A may be determined by the impedance of sacrificial link 43. Voltage-sensing block 110 senses the voltage V at node A and an analog-to-digital converter in MCU 80 senses the voltage V.

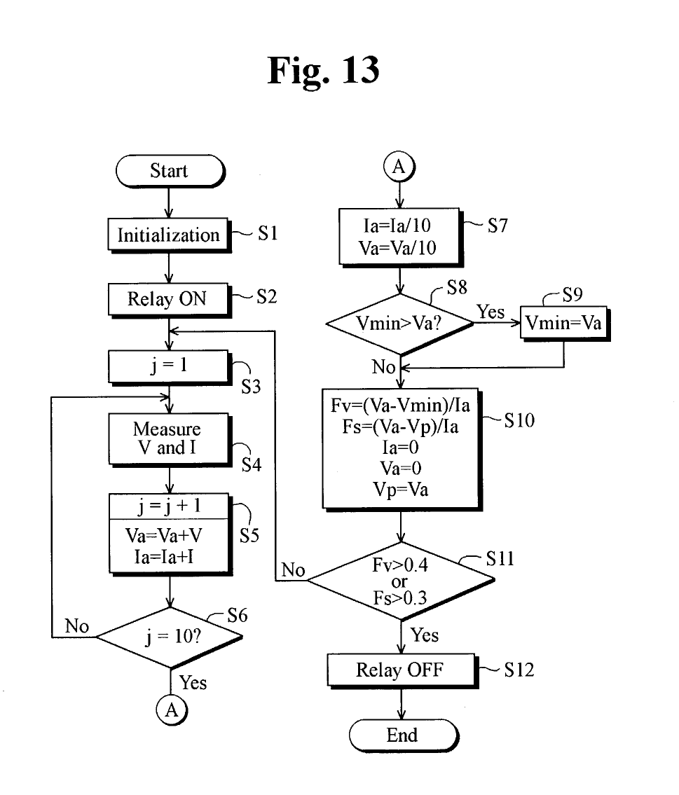

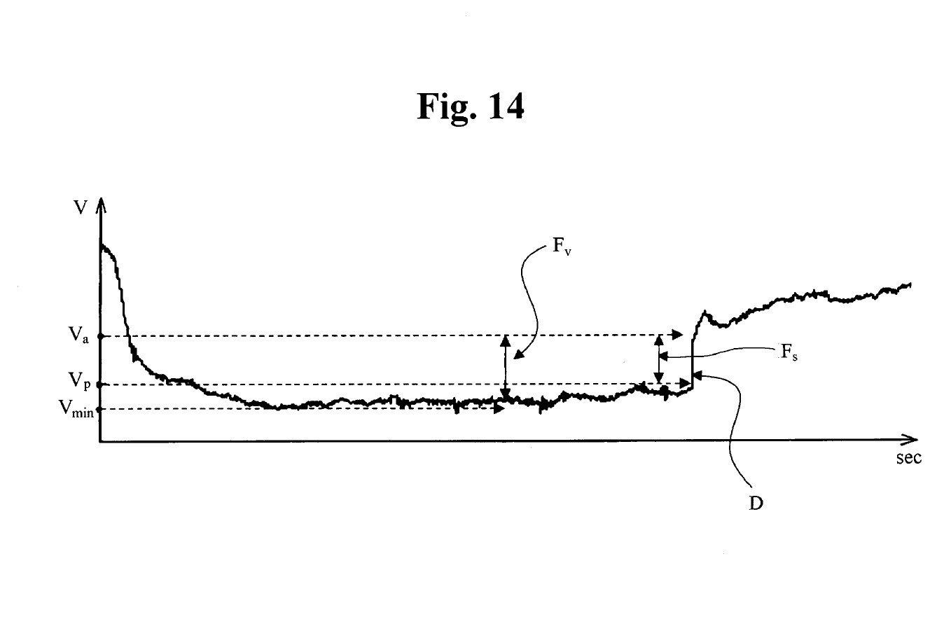

FIG. 13 is a flow chart, which will be used to describe a method for detecting the detachment of an embolic material in accordance with the present invention. FIG. 14 is a graphical representation illustrating a change in the voltage of sacrificial link 43 during the electrolysis.

In the following, the inventive method will be described in detail in conjunction with FIGS. 9 and 11 to 14.

At step S1, system 500 of the present invention is initialized. Next, relay 120 is activated at step S2. As mentioned above, the conductive state of relay 120 forms a closed loop consisting of system 500 and patient 130. In FIG. 11, t₁ represents the time at which current power is applied to guiding wire 41 inside patient 130, and (X represents a unique current value of patient 130 at time ti. The current value is gradually increased up to 1˜2 mA for a predetermined time period (i.e., t₁ to t₂), as shown in FIG. 11.

Once power is applied to guiding wire 41, the voltage between the proximal end of guiding wire 41 and the body of patient 130 or the ground is measured a plurality of times, e.g., 10 times per second. Likewise, the current flowing to guiding wire 41 is measured the same number of times.

At step S7, average Ia of the current measurements and average Va of the voltage measurements are computed and stored in a memory 140 shown in FIG. 9. This averaging would be performed for another set of a plurality of measurements until the following steps verify the detachment of embolic material 45.

At step S8, voltage average Va obtained at step S7 is compared with minimum average Vmin. Here, the minimum average is the smallest of all the averages of sets of measurements taken so far, excluding the present average voltage. If the present average voltage is smaller than the minimum average, it is replaced with the present average voltage before next step S10 is performed. In other words, the present average voltage becomes minimum average Vmin from the perspective of the next cycle of voltage measurements.

At step S10, a fluctuation in the impedance of sacrificial link 43 is calculated based on minimum voltage Vmin, present average voltage Va, and present average current Ia by using the following equations,

$$\begin{matrix}Fv = \frac{Va - V min}{Ia} & Eq . ( 1 ) \\ Fs = \frac{Va - Vp}{Ia} & Eq . ( 2 )\end{matrix}$$

wherein Fv and Fs represent a fluctuation in the impedance of sacrificial link 43, respectively, and Vp represents the average of the previous measurements of voltage, as shown in FIG. 14.

After the impedance fluctuation is calculated by using the above Eqs. (1) and (2), present average Va would be stored as previous average Vp (“previous” from the perspective of the next average voltage of new voltage measurements). At step S11, if Fv is greater than a first predetermined threshold or Fs is greater than a second predetermined threshold, sacrificial link 43 is determined to have been detached. Then, relay 120 is turned off to a non-conductive state. Otherwise, another plurality of measurements is taken to compute new present average voltage and current. More specifically, at step S11, in case that the first and second predetermined thresholds are, e.g., 0.4 and 0.3, respectively, a relation Fv>0.4 represents that sacrificial link 43 has been gradually disconnected, and a relation Fs>0.3 represents that sacrificial link 43 has been suddenly disconnected. For example, FIG. 11 represents an illustrative case where sacrificial link 43 has been suddenly detached, which shows that sacrificial link 43 begins to be electrolyzed at time t₃ and is detached at time t₄. In short, the total time taken to electrolyze sacrificial link 43 is from t₁ to t₄ and the total time taken for the disappearance of sacrificial link 43 is from t₃ to t₄.

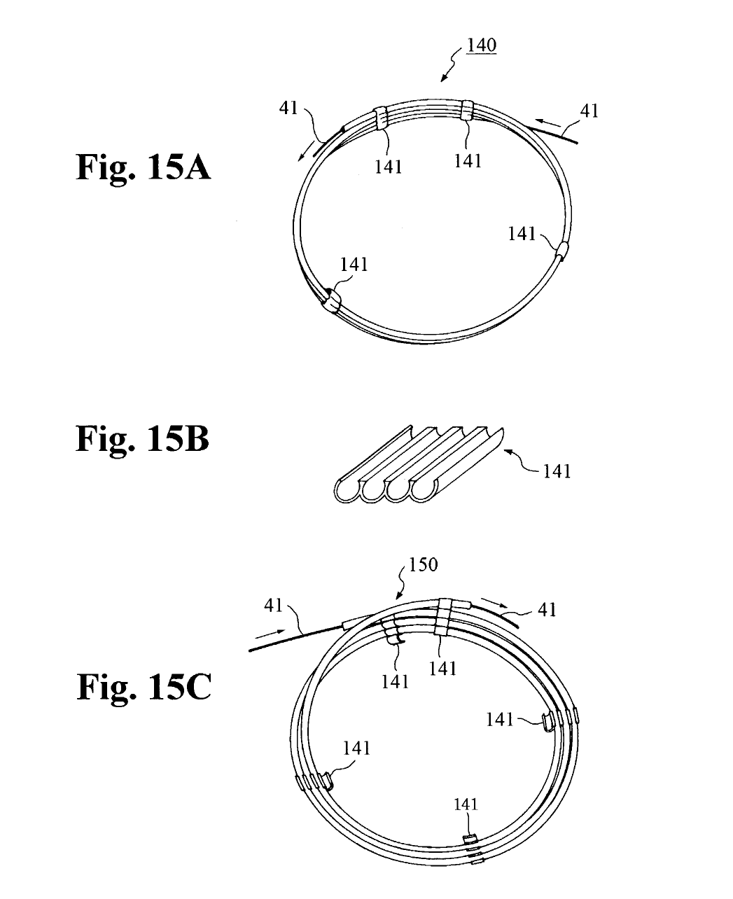

FIGS. 15A and 15B are various types of tubes used in keeping therein the inventive assembly for embolization in accordance with the present invention. As shown in FIG. 15A, a tube 140 is tailored to have a length and an internal diameter sufficient to keep the inventive assembly therein and is rolled in a preset diameter. Tube 140 is made of Polyethylene. A multiplicity of clips 141 is also disposed at certain intervals on a tube 140 to maintain the rolling. The multiplicity of clips 141 is designed in a one side-opened form to permit tube 140 to be plucked out of them. 46

As shown in FIG. 15A, tube 140 is rolled such that two ends are on a substantially straight line. This allows the rather easy insertion of embolic material 45 into vascular malformation 11. That is, the operator aligns the outlet end of tube 140 to the implant of micro catheter 10 (shown in FIGS. 2A and 2B), and holds and pushes guiding wire 41 at the side of the implant end of tube 140 so that the outlet end of guiding wire 41 is inserted into the implant of micro catheter 10 toward vascular malformation 11. FIG. 15B is a pictorial view depicting clips 141. As shown in FIG. 15C, a tube 150 may be concentrically rolled using clips 141 of FIG. 15B.

As previously mentioned, the present invention employs an embolic material into which a Platinum wire with a good conductivity is inserted to thereby enhance a column strength of the embolic material and effectively increase a thrombus rate without any application of high power to the embolic material. Furthermore, the present invention employs a guiding member having various tapered portions thereon and a specific tailored tube for housing therein the guiding member and the embolic material, to allow the embolic material to be easily inserted into vascular malformations such as cerebral aneurysms.

Moreover, the present invention employs a micro-envelope tailored to surround a minute stepped portion at which a connection portion and the embolic material are coupled, to thereby allow ingress and egress of the embolic material in a distal end of a micro catheter to be easy and improve the imaging ability of the embolic material under fluoroscopy. In addition, in contrast with a conventional apparatus using DC power with AC superposition, the present invention employs a single DC power supply, to thereby simplify the structure thereof and lower production cost. Likewise, the present invention automatically senses minute fluctuations in the DC impedance of the sacrificial link to thereby exactly detect the instant that the embolic material is detached from the guiding member.

While particular embodiments of the present invention have been shown and described, it will be obvious to those skilled in the art that changes and modifications may be made without departing from this invention in its broader aspects and, therefore, the aim in the appended claims is to cover all such changes and modifications, as fall within the true spirit and scope of this invention.

What is claimed is:

1. An assembly for use in occluding a target site in a living being, which comprises:
an implant to be inserted within the target site; and
a guiding member with its distal end coupled with the implant, for guiding the implant to the target site, wherein the guiding member is made of an electrically conducting material and includes a multiplicity of tapered configurations.

2. The assembly of claim 1, wherein the implant includes a coil configuration through which an electrically conducting wire is passed to enhance a thrombus in the target site.

3. The assembly of claim 2, wherein one end of the implant includes a semicircle-like welded portion formed by melting the wire and the other end of the implant is coupled with the distal end of the guiding member.

4. The assembly of claim 3, wherein the other end of the implant has an internal diameter sufficient to accommodate the distal end of the guiding member and the electrically conducting wire therein.

5. The assembly of claim 4, wherein the other end of the implant and the distal end of the guiding member are coupled from each other by micro spot welding.

6. The assembly of claim 4, wherein the electrically conducting wire is made of Platinum.

7. The assembly of claim 5, wherein the guiding member is made of stainless steel and the surface of the guiding member is coated with a PTFE (Poly Teflon Fluorine Ethylene) material.

8. The assembly of claim 5, wherein the implant is made of one of Tungsten and an Iridium alloy with a main component of Platinum.

9. The assembly of claim 1, further comprising a tube, made of a polyethylene, for keeping the implant and the guiding member therein to facilitate the insertion of the implant into the target site.

10. The assembly of claim 1, further comprising a micro envelope for surrounding the implant and the distal end of the guiding member.

11. The assembly of claim 10, wherein the micro envelope is made of one of a pure radiopaque Gold and a Platinum alloy with a thickness of less than 10 μm and a length of 0.3 mm.
